(19) United States
(12) Patent Application Publication
(10) Pub. No.: US 2008/0046018 A1
(43) Pub. Date: Feb. 21, 2008

(54) SWITCH FOR MEDICAL DEVICE

(75) Inventors: Nicholas Oliver Von Huben, Bexley North (AU); Peter Joseph Ayre, Crows Nest (AU); Lee Thomas Glanzmann, Redfern (AU)

Correspondence Address: MCCARTER & ENGLISH , LLP STAMFORD OFFICE, FINANCIAL CENTRE , SUITE 304A, 695 EAST MAIN STREET, STAMFORD, CT, 06901-2138, US

(73) Assignee: VENTRASSIST PTY LTD, Chatswood (AU)

(21) Appl. No.: 11/839,117

(22) Filed: Aug. 15, 2007

(30) Foreign Application Priority Data
Aug. 16, 2006 (AU) 2006904454

(51) Int. Cl. A61N 1/08

(52) U.S. Cl. 607/27

(57) ABSTRACT

A switch for use with a medical device. The switch includes an accelerometer for detecting motion, a controller cooperating with said accelerometer to determine whether said detected motion is substantially identical to a predefined motion of a user. The controller alters the functioning state of the medical device, if the detected motion is substantially identical to the predefined motion of the user.

### FIELD OF THE INVENTION

The present invention relates to improvements to switches and systems thereof. Preferably, the present invention relates to improvement to switches for use with implanted medical devices.

### BACKGROUND OF THE INVENTION

Previously, switches have relied on a user mechanically joining two electrical contacts together to form a circuit to operate a device. These types of mechanical switches are not suitable for all applications. In particular, implanted medical device applications wherein the switch may be located beneath the skin layer of an implanted patient.

In some environments, electrical contacts may corrode and deteriorate. Also it may be difficult to mount switches in some environments. Contact type switches are generally not suitable for implantation.

U.S. Pat. No. 5,317,305—Campman describes a prior art switch wherein a single axis accelerometer is used to operate a personal alarm. When the device is vibrated, an integrated audible alarm is activated. This device does not disclose or teach that accelerometers may be used or should be used with medical devices, implantable or otherwise. Additionally, it fails to teach that multiple axis accelerometers will function better when identifying a user motion which is intended to selectively operate the device and thereby the use of the single axis accelerometer reduces specificity of the device.

U.S. Pat. No. 5,609,614—Stotts et al describes an implantable cardiac simulator connected to a accelerometer, wherein the accelerometer detects movements in muscle tissue due to over or under simulation. The user is this embodiment fails to have any control over the switching process. Furthermore, the accelerometer described relates only to a single axis accelerometer.

The present invention aims to or at least address or ameliorate one or more of the disadvantages associated with the above mentioned prior art.

### SUMMARY OF THE INVENTION

According to a first aspect the present invention consists in a switch for operating a medical device wherein said switch includes: an accelerometer for detecting motion; a controller cooperating with said accelerometer to determine whether said detected motion is substantially identical to a predefined motion of a user and wherein the controller alters the functioning state of the medical device, if the detected motion is substantially identical to the predefined motion of the user.

Preferably said functioning state is either on or off.

Preferably the accelerometer has more than a single axis.

Preferably the accelerometer is implanted within the user.

Preferably the device is a medical device implanted within the user.

According to a second aspect the present invention consists in a system for operating a medical device wherein said system includes: detecting a motion of a user; comparing said detected motion to a predefined motion of a user and wherein the functioning state of the medical device is altered, if the detected motion is substantially identical to the predefined motion of the user.

According to a third aspect the present invention consists in a switch for use with an implantable medical device, said switch adapted to be disposed under the skin layer of a patient, said switch operable by a slapping motion of said patient.

Preferably said switch comprises an accelerometer for detecting motion operably connected to a controller for determining whether a motion detected by said accelerometer is substantially identical to a predefined slapping motion of said patient, and wherein said controller alters the functioning state of said medical device if said detected motion is substantially identical to said predefined slapping motion.

According to a fourth aspect the present invention consists in an implantable medical device comprising a switch adapted to be disposed under the skin layer of a patient, said switch operable by a slapping motion of said patient.

Preferably said switch comprises an accelerometer for detecting motion operably connected to a controller for determining whether a motion detected by said accelerometer is substantially identical to a predefined slapping motion of said patient, and wherein said controller alters the functioning state of said medical device if said detected motion is substantially identical to said predefined slapping motion.

According to a fifth aspect the present invention consists in a switch for operating a medical device wherein said switch includes: an accelerometer for detecting motion; a controller cooperating with said accelerometer to determine whether said detected motion is substantially identical to a predefined motion of a user recorded in a memory device integral with or connected to said controller, and wherein the controller alters the functioning state of the medical device, if the detected motion is substantially identical to the predefined motion of the user.

### BRIEF DESCRIPTION OF THE DRAWINGS

Embodiments of the present invention will now be described with reference to the accompanying drawings wherein:

FIG. 1 depicts a schematic view of a first embodiment of the present invention; and

FIG. 2 depicts a further enlarged schematic view of the first embodiment of the present invention.

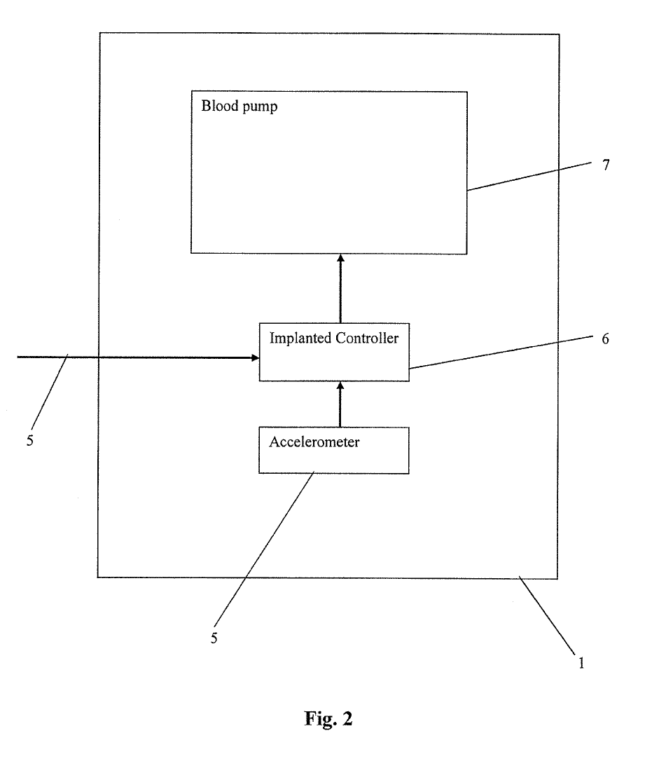

### DESCRIPTION OF THE PREFERRED EMBODIMENTS

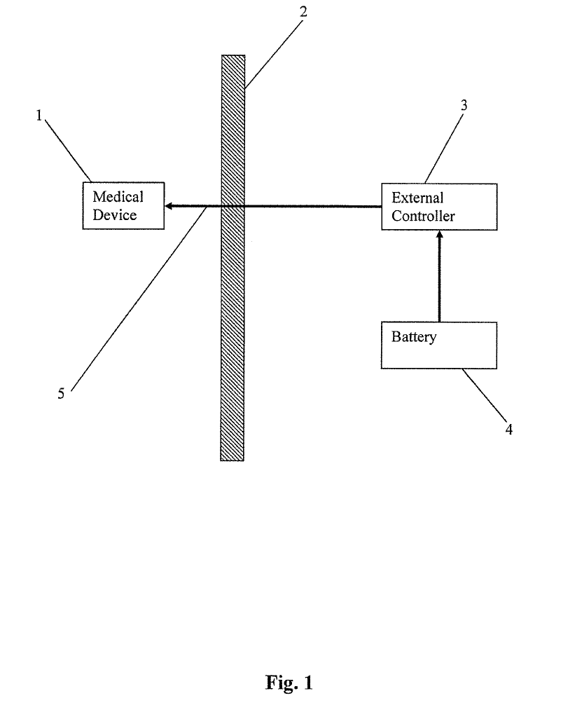

In a first embodiment of the present invention as shown in FIGS. 1 & 2, an implanted medical device 1 is positioned beneath the skin layer 2 of a patient. The implanted medical device 1 is preferably controlled and/or powered by external means. In this embodiment, the external means comprises external controller 3 joined to implanted medical device 1 by percutaneous lead 5. External controller 3 is preferably powered by at least one battery 4.

Preferably the implanted medical device 1 includes: a blood pump 7; an implanted controller 6 and an accelerometer 5. The blood pump 7 is connected to the circulatory system of the patient to provide circulatory support. One preferred use of the blood pump 7 may be to connect it in parallel with the normal flow of heart between the apex of the left ventricle and ascending aorta, and this may specifically offload the left side of the heart and improve cardiac function.

Preferably, the implanted controller 6 is connected to the blood pump 7 and is capable of controlling and driving the blood pump 7. The implanted controller 6 is also connected to accelerometer 5.

The accelerometer 5 is adapted to detect sudden motion or impacts. Preferably, the accelerometer 5 is mounted and positioned in such a manner as to allow a patient implanted with the medical device 1 to be able to slap the local area on their skin layer 2, near to where the medical device 1 is implanted. The sudden motion caused by the action of the slap may be detected by the accelerometer 5.

Preferably, the implanted controller 6 may be able to differentiate between the patient's normal motion which is similar to background motion and the intentional slapping motion both of which are detected by the accelerometer 5. After the implanted controller 6 determines whether the sudden motion was intentional, it may then alter a functioning state of the blood pump 7. The derivation of the detected motion may be accomplished by the use of a comparing algorithm within the implanted controller 6 which compares the detected motion against pre-recorded detected motions of a slap.

Preferably, if the implanted controller 6 determines that the detected motion is substantially identical to the predefined or pre-recorded motion, the implanted controller 6 may operate a latch type circuit and switch the functioning state of the blood pump 7. Preferably, the implanted controller 6 may amend the functioning state of the blood pump 7 by: changing the pumping speed to other predetermined speeds; or turning the pump on or off.

Preferably, the predefined (or pre-recorded) motion has been recorded in a memory device integral with or connected to controller 6.

Preferably, the first embodiment of the present invention may work with left ventricle assist devices such as the device described within U.S. Pat. No. 6,227,797—Watterson et al. This particular device is generally a centrifugal blood pump with a hydrodynamically borne or suspended impeller. This device connects between the apex of the left ventricle and the ascending aorta of the implanted patient.

The accelerometer 5 may preferably be a tri-axial accelerometer. At least a single axis accelerometer may be sufficient to function with the first embodiment to detect the desired motion. Additionally, digital or analogue accelerometers may also work with this first embodiment of the present invention.

Alternately in the first embodiment, other power sources may be used to replace the battery 4 and these power sources may include mains power connection and the like. Furthermore even implanted power sources may be used with the first embodiment. Additionally, the implanted controller 6 may include a power source (not shown) such as an additional battery to supply power in the event of failure of the external controller 3. Also, the percutaneous lead 5 may be replaced with a system that does not require the skin layer 2 of the patient to be breached. This type of system is well known and covered extensively in prior art patents relating to transcutaneous energy transmission systems or TETS.

A further improvement beyond the first embodiment of the present invention may also be to include within the implanted controller 6 all of the features of the external controller 3 and thereby removing the need for the external controller 3. This integrated design or improvement may be lighter and less bulky for a patient to carry.

Preferably, either controller may preferably transmit and receive data wirelessly with other computing systems and networks. This may be accomplished using Bluetooth™ or other wireless protocols. The controllers may store and log data and information relating to the functioning state of the medical device 1 and the detected motions of the patient. Furthermore, the first embodiment of the present invention may be suitable for use with other systems and medical devices. Whilst the preferred systems for use are left ventricle assist systems other systems may include, but not limited to: neural simulators, cochlear implants and pacemakers.

In a further embodiment, the external controller 3 may include the accelerometer 5 rather than the internal controller 6. The accelerometer 5, in this embodiment, may function in a similar manner to the earlier described embodiment.

Preferably, the user or patient, using the medical device 1, may strike the casing or housing of the external controller 3. The accelerometer 5 integrated into the external controller 3 may then detect the striking motion or impact and the external controller 3 may then determine whether the striking motion matches a predetermined and pre-recorded motion. If the striking motion is sufficiently similar to the predetermined motion, the external controller 3 may then instruct the medical device 1 to change its functioning state. In the situations where the medical device 1 is an implanted blood pump, the external controller 3 may instruct the blood pump to change pumping speeds.

Additionally, the external controller 3 may be modified to determine several different predetermined motions. The external controller 3 may for instance, respond to a series of two consecutive strikes by the user, thereby instructing the external controller 3 to change the pumping speed of the blood pump to a predetermined level. Three consecutive strikes may instruct the external controller 3 to change the pumping speed to a second determined level. A person skilled in the art may appreciate that any number of determined motions could be used to instruct the external controller 3 to change the appropriate functioning state of the medical device 1.

Furthermore, the accelerometer 5 may be used to detect other predetermined motions including shaking or changes in patient orientation such as a patient laying on their side or back. The functioning state of the medical device 1 may be adjusted in accordance with the motion.

The above descriptions detail only some of the embodiments of the present invention. Modifications may be obvious to those skilled in the art and may be made without departing from the scope and spirit of the present invention.

1. A switch for operating a medical device wherein said switch includes: an accelerometer for detecting motion; a controller cooperating with said accelerometer to determine whether said detected motion is substantially identical to a predefined motion of a user and wherein the controller alters the functioning state of the medical device, if the detected motion is substantially identical to the predefined motion of the user.

2. The switch as claimed in claim 1, wherein said functioning state is either on or off.

3. The switch as claimed in claim 1, wherein the accelerometer has more than a single axis.

4. The switch as claimed in claim 2, wherein the accelerometer is implanted within the user.

5. The switch as claimed in claim 2, wherein the device is a medical device implanted within the user.

6. A system for operating a medical device wherein said system includes: detecting a motion of a user; comparing said detected motion to a predefined motion of a user and wherein the functioning state of the medical device is altered, if the detected motion is substantially identical to the predefined motion of the user.

7. A switch for use with an implantable medical device, said switch adapted to be disposed under the skin layer of a patient, said switch operable by a slapping motion of said patient.

8. A switch as claimed in claim 7, wherein said switch comprises an accelerometer for detecting motion operably connected to a controller for determining whether a motion detected by said accelerometer is substantially identical to a predefined slapping motion of said patient, and wherein said controller alters the functioning state of said medical device if said detected motion is substantially identical to said predefined slapping motion.

9. An implantable medical device comprising a switch adapted to be disposed under the skin layer of a patient, said switch operable by a slapping motion of said patient.

10. An implantable medical device as claimed in claim 9, wherein said switch comprises an accelerometer for detecting motion operably connected to a controller for determining whether a motion detected by said accelerometer is substantially identical to a predefined slapping motion of said patient, and wherein said controller alters the functioning state of said medical device if said detected motion is substantially identical to said predefined slapping motion.

11. A switch for operating a medical device wherein said switch includes: an accelerometer for detecting motion; a controller cooperating with said accelerometer to determine whether said detected motion is substantially identical to a predefined motion of a user recorded in a memory device integral with or connected to said controller, and wherein the controller alters the functioning state of the medical device, if the detected motion is substantially identical to the predefined motion of the user.
